(19) United States
(12) Patent Application Publication
(10) Pub. No.: US 2020/0214950 A1
(43) Pub. Date: Jul. 9, 2020

(54) MATTE LIPSTICK COMPOSITION

(71) Applicant: CHANEL PARFUMS BEAUTE, Neuilly Sur Seine (FR)

(72) Inventors: Hélène FIOLEAU, Paris (FR); Hélène DE CLERMONT-GALLERANDE, Vincennes (FR); Chantal MALVEZIN, Lamorlaye (FR)

(73) Assignee: CHANEL PARFUMS BEAUTE, Neuilly Sur Seine (FR)

(21) Appl. No.: 16/647,964

(22) PCT Filed: Oct. 5, 2018
(86) PCT No.: PCT/FR2018/052458

§ 371 Date: Mar. 17, 2020

(30) Foreign Application Priority Data
Oct. 5, 2017 (FR) 17 59358

(51) Int. Cl. A61K 8/25; A61Q 1/06; A61K 8/02; A61K 8/92

(52) U.S. Cl. CPC A61K 8/25; A61Q 1/06; A61K 2800/43; A61K 8/92; A61K 2800/10; A61K 8/0229

(57) ABSTRACT

A matte lipstick composition, including at least one structure-directing agent, at least one oil, at least one colouring agent, and at least one particular filler as a mattifying agent, characterised in that the particular filler serving as a mattifying agent is selected from among particles having a specific surface area of less than 30 m²/g, preferably less than 20 m²/g, more preferably still less than 10 m²/g, and advantageously less than 5 m²/g. Also a lipstick including the lipstick composition, a method for producing said lipstick composition, and the use of a particular filler in order to mattify a lipstick composition without hardening same.

### TECHNICAL FIELD OF THE INVENTION

The invention relates to the field of cosmetic products, and more particularly matte and colour-intense solid cosmetic compositions such as matte and intense lipsticks.

### TECHNICAL BACKGROUND

The use of lipsticks as means for colouring lips has undergone rapid growth, such that lipsticks are now widespread beauty products. A cosmetically acceptable lipstick should spread easily, have a homogeneous colour and exhibit a melting point above body temperature. Moreover, it should give the lips a smooth but non-greasy appearance and should retain the consistency thereof without exudation, oozing, rupture or disintegration phenomena occurring.

A lipstick should not only have the qualities described above, but above all give the lips a smooth and creamy sensation and protect them from drying or chapping. It is particularly difficult to obtain such a smooth and creamy sensation using lipsticks having a reduced sheen or gloss, namely a matte appearance.

Routinely, matte lipsticks contain various clays and silicas with a view to obtaining a matte appearance. The use of clays and silicas results in a hard stick which becomes increasingly dry and hard when the solid substance content increases. Moreover, on applying such a matte lipstick on the lips, a sensation of dryness is obtained. This results therefore in a certain need to produce a lipstick providing a creamy application while allowing a matte appearance, which up to now was indissociable with a dry texture.

The first aim of the present invention is that of providing a lipstick having a matte and colour-intense appearance and a homogeneous and light texture and which remains so over time.

The present invention is characterised in that the matte lipstick composition comprises at least one structuring agent, at least one oil, at least one colouring agent, and at least one particular filler as a mattifying agent.

Thus, the invention relates according to a first aspect, to a matte lipstick composition comprising at least one structuring agent, at least one oil, at least one colouring agent, and at least one particular filler as a mattifying agent, characterised in that the particular filler serving as a mattifying agent is selected from among particles having a specific surface area of less than 30 m²/g, preferably less than 20 m²/g, more preferably still less than 10 m²/g and even more advantageously less than 5 m²/g.

The invention also relates, according to a further aspect, to a matte lipstick composition comprising at least one structuring agent, at least one oil, at least one colouring agent, and at least one particular filler as a mattifying agent, characterised in that the particular filler serving as a mattifying agent is selected from among magnesium carbonate powders having a specific surface area of less than 10 m²/g and even more advantageously less than 5 m²/g.

Finally, the invention further relates, according to a third aspect, to a matte lipstick composition comprising at least one structuring agent, at least one oil, at least one colouring agent, and at least one particular filler as a mattifying agent, characterised in that the particular filler serving as a mattifying agent is selected from among magnesium carbonate powders, said lipstick composition according to the invention spread on a contrast chart having a gloss value measured at 85° using a glossmeter known as micro-TRI-gloss or PICOGLOSS 503 sold by the company BYK of less than 10, and said composition having a hardness measured at 20° C. using a texture analyser sold under the trade name “TA-XT Plus Microstable System” by the company Swantech of less than 300 g (grams), 1 day after manufacture and after storage of 1 day, 15 days, 1 month and 2 months in an oven at 45° C.

### The Particular Filler Used as a Mattifying Agent

Filler denotes solid particles intended to be dispersed in the medium of the composition and which remain insoluble in this medium, regardless of the temperature at which the composition is manufactured and stored.

The particular filler used as a mattifying agent according to the invention is a particle characterised by the size, shape and specific surface area thereof.

The size of the particular filler used measured taking the most distant extremities of the particle is between 1 micron (μm) and 30 microns (μm). The smaller the size thereof, the more matte the finish thereof.

The shape of the particular filler used is lamellar or cubic or spherical or rounded if it is less than 10 microns in size. On the other hand, when the size thereof is greater than 10 microns, the shape thereof is preferably spherical or rounded to prevent specular reflection and therefore a satiny or gloss effect.

The specific surface area of the particular filler is less than 30 m²/g, preferably the specific surface area is less than 20 m²/g, even more preferably the specific surface area is less than 10 m²/g and even more advantageously the specific surface area is less than 5 m²/g.

The specific surface area measurements of the mattifying fillers may be carried out using a BET tristar II measuring apparatus. The method is based on the adsorption of nitrogen molecules on the surface and in the pores of the powder sample at low temperature (nitrogen liquefaction temperature: 77° K). The specific surface area is expressed in m²/g.

Among the particular fillers suitable for use in the present invention, mention may be made of magnesium carbonate. More specifically, the particular filler is chosen from among magnesium carbonate powders, in particular basic magnesium carbonate of formula (MgCO₃)₃.Mg(OH)₂.3H₂O or of formula (MgCO₃)₄.Mg(OH)₂.5H₂O or indeed normal magnesium carbonate of formula (MgCO₃).xH₂O. Mention may be made, by way of example, of the magnesium carbonate sold by the company ICL Industrial products under the name “basic magnesium carbonate”. The particles are present at size between 5 microns and 25 microns, they have a desert rose shape and the specific surface area thereof is about 4 m²/g.

Among the particular fillers suitable for use in the present invention, mention may also be made of diatomaceous earths. Mention may be made in particular of the diatomaceous earths sold by the company Imerys under the name Imercare® 03D diatomaceous earth. The particles are present at sizes between 2 microns and 5 microns, they have various shapes and the specific surface area thereof is about 12 m²/g.

Mention may also be made of the diatomaceous earths marketed by the company Imerys under the name “Imercare® 400D diatomaceous earth”. The particles are present at sizes between 15 microns and 25 microns, they have various shapes and the specific surface area thereof is about 19 m²/g.

Among the particular fillers, mention may be made of microcrystalline cellulose. Mention may particularly be made of that marketed by Rettenmaier under the name Vivapur® CS 4FM. The particles are present at sizes between 0.9 microns and 9.4 microns and the specific surface area thereof is about 5.7 m²/g.

Among the particular fillers, mention may also be made of a bamboo powder. Mention may made of that marketed by Rossow under the name Sencocel® BC20.

The particular fillers chosen from basic magnesium carbonate, diatomaceous earths, microcrystalline cellulose, or one of the mixtures thereof, are preferred.

The particular filler used as a mattifying agent may be used as is or indeed may be surface-treated with a hydrophobic agent.

Among the hydrophobic agents, mention may particularly be made of silanes, silicones, fatty acid soaps, C₉₋₁₅ fluoroalcohol phosphates, acrylate/dimethicone copolymers, mixed C₉₋₁₅ fluoroalcohol phosphate/silicone copolymers, lecithins, carnauba wax, polyethylene, chitosan and optionally acylated amino acids such as lauroyl lysine, disodium stearoyl glutamate and aluminium acyl glutamate.

According to the invention, the particular filler used as a mattifying agent is present in a proportion of between 8% and 20% and preferably between 10% and 15% by weight with respect to the total weight of the composition.

### The Oil

According to the present invention, “oil” denotes a liquid compound at ambient temperature (25° C.), and which, when introduced at a rate of at least 1% by weight into water at 25° C., is not at all soluble in water, or soluble at a rate of less than 10% by weight, with respect to the weight of oil introduced into the water.

In the context of the present invention, the term “oil” also comprises lipophilic fats which are capable of undergoing a reversible liquid/solid change and have an anisotropic crystalline organisation in the solid state, but which are different from waxes in that they contain, at a temperature of 23° C., a liquid fraction and a solid fraction. A compound of this type is particularly a mixture of sterol esters, such as the cholesterol and lanosterol ester mixture available from the manufacturer CRODA under the trade name Super Sterol Ester®.

Non-Volatile Oil

“Non-volatile oil” denotes an oil which has a boiling point generally greater than 300° C. under 760 mm of Hg (101325 Pa) and which has little or no vapour tension.

The non-volatile oils may particularly be chosen from among non-volatile silicon oils, non-volatile hydrocarbon oils, and mixtures thereof.

“Silicone oil” denotes an oil comprising at least one silicon atom, and particularly at least one Si—O group.

As a non-volatile silicone oil, mention may particularly be made of polydimethylsiloxanes containing at least 8 silicon atoms, polyalkylmethylsiloxanes wherein the alkyl chain contains from 8 to 20 carbon atoms and the oils identified by the name INCI name phenyl trimethicone.

“Hydrocarbon oil” denotes an oil containing only hydrogen and carbon atoms.

Mention may be made for example of hydrocarbons such as squalane, phytosqualane, polybutene, hydrogenated polyisobutene, hydrogenated polydecene, synthetic (poly) esters also known as “ester oils” and (poly) ethers, in particular C6-C20 acid and C6-C20 alcohol (poly) esters, advantageously branched such as isononyl isononanoate; plant oils; branched and/or unsaturated fatty acids; branched and/or unsaturated fatty alcohols such as octyldodecanol; or one of the mixtures thereof.

“Ester oil” denotes a mono-, di-, tri- or tetra-ester oil. The ester oils are obtained by reacting a mono-, di-, tri- and more generally a polyol with a mono- di- tri- and more generally a carboxylic polyacid, said reagents optionally being linear or branched, saturated or unsaturated, aliphatic or aromatic, and optionally comprising alkoxylated groups. The ester oils may particularly be hydroxylated.

In particular, the non-volatile ester oil may comprise from 18 to 70 carbon atoms.

The non-volatile ester oil may particularly be chosen from among:
monoesters comprising 18 to 40 carbon atoms, in particular monoesters of formula R1COOR2 wherein R1 represents the residue of a linear or branched fatty acid comprising from 6 to 20 carbon atoms and R2 represents a hydrocarbon chain, particularly branched, containing from 6 to 20 carbon atoms, such as for example Purcellin oil (cetostearyl octanoate), isononyl isononanoate, isodecyl neopentanoate, C12 to C15 alkyl benzoates, 2-ethylhexyl palmitate, octyldodecyl neopentanoate, 2-octyldodecyl stearate, 2-octyldodecyl erucate, isostearyl isostearate, 2-octyldodecyl benzoate, alkyl octanoates, decanoates or ricinoleates, isopropyl myristate, isopropyl palmitate, butyl stearate, hexyl laurate, 2-ethylhexyl palmitate, 2-hexyldecyl laurate, 2-octyldecyl palmitate, 2-octyldodecyl palmitate, 2-diethylhexyl succinate;
diesters comprising 18 to 60 carbon atoms, in particular from 18 to 50 carbon atoms, such as diesters of carboxylic acid and monoalcohols, such as diisostearyl malate; diesters of glycol and carboxylic monoacids, such as neopentylglycol diheptonoate and polyglyceryl-2 diisostearate;
triesters comprising 35 to 70 carbon atoms, such as triesters of carboxylic triacid, such as triisostearyl citrate or tridecyl trimellitate; or triesters of glycol and carboxylic monoacids such as polyglyceryl-2 triisostearate;
tetraesters comprising 35 to 70 carbon atoms, such as such as tetraesters of penthaerythritol or polyglycerol and a carboxylic monoacid, for example pentaerythrityl tetrapelargonate, pentaerythrityl tetraisostearate, pentaerythrityl tetraisononanoate, glyceryl tridecyl-2 tetradecanoate, polyglyceryl-2 tetraisostearate or indeed pentaerythrityl tetradecyl-2 tetradecanoate;
polyesters obtained by condensation of dimer and/or trimer of unsaturated fatty acid and diol such as those described in patent application FR 0 853 634, such as the polyester of dilinoleic acid and 1,4-butanediol;
esters and polyesters of dimer diol and mono- or dicarboxylic acid, such as the esters of dimer diol and fatty acid and the esters of dimer diols and carboxylic diacid dimer, in particular those obtained from a dimer of a C8 to C34, particularly C12 to C22, in particular C16 to C20, and more particularly C18 unsaturated fatty acid, such as the esters of dilinoleic diacids and dilinoleic dimer diols, for example those marketed by the company NIPPON FINE CHEMICAL under the trade name LUSPLAN DD-DA5® and DD-DA7®;
triglycerides of fatty acids (liquid at ambient temperature), particularly of fatty acids having from 7 to 40 carbon atoms, such as heptanoic or octanoic acid triglycerides or jojoba oil; saturated triglycerides such as caprylic/capric triglyceride, glyceryl triheptanoate, glycerin trioctanoate; C18-36 acid triglycerides such as those marketed under the reference DUB TGI 24 marketed by Stéarineries Dubois); and unsaturated triglycerides such as castor oil, olive oil, ximenia oil, pracaxi oil;
or one of the mixtures thereof.

The non-volatile oil used in the present invention is preferably a low-gloss oil.

A low-gloss oil denotes an oil wherein the refractive index is less than 1.46, preferably less than 1.45, more preferably less than 1.44.

The refractive index is measured using an ABBE paralux ref 60-6400-9 refractometer.

Among the low-gloss oils suitable for use in the invention, mention may be made for example of synthetic (poly) esters and (poly) ethers, in particular (poly) esters of C6-C20 acids and C6-C20 alcohols, advantageously branched, such as isononyl isononanoate; di(C6-C20 alkyl) carbonates such as dicaprylyl carbonate marketed by BASF under the trade name Cetiol CC; branched and/or unsaturated fatty acids; polyesters of polyols, in particular of (di) pentaerythritol, such as pentaerythritol tetraoctanoate, silicone oils such as linear polydimethylsiloxanes of viscosity between 5 cSt and 100 cSt (centistokes); or one of the mixtures thereof.

Among the low-gloss oils suitable for use in the invention, low-viscosity oils are preferably used. Low-viscosity denotes a viscosity of less than 1000 Pa·s, preferably less than 500 Pa·s, more preferably less than 100 Pa·s and even more advantageously less than 50 Pa·s.

The viscosity is measured using a Gemini rheometer sold by the company Malvern Instruments. A measurement is carried in stress sweep rotation from 0.01 Pa to 1000 Pa. A bob system is used with coaxial cylinders of 25 mm in diameter (C25) with an air-gap of 150 microns at a temperature of 20° C. The value indicated for each oil is the mean obtained on several points of the plateau.

Among these low-gloss and low-viscosity oils, mention may be made of isononyl isononanoate, dicaprylyl carbonate or one of the mixtures thereof.

This makes it possible indeed to incorporate more particular filler as a mattifying agent and more pigments and therefore obtain a colour that is both matte and intense.

An additional non-volatile oil may also be used to add further properties to the composition according to the invention.

By way of example, diisostearyl malate may be added as it makes it possible to obtain good pigment dispersion.

Further additional oils may be added to enhance the sensory properties of the formula.

According to an embodiment, the non-volatile oil is chosen from among isononyl isononanoate, dicaprylyl carbonate, diisostearyl malate or one of the mixtures thereof.

According to a particular embodiment of the invention, the oil introduced into the composition according to the invention is a mixture of isononyl isononanoate, dicaprylyl carbonate and diisostearyl malate.

The oil may be present in the composition according to the invention at a content of between 40% and 80% by weight, preferably between 45% and 70% by weight, more preferentially between 50% and 60% by weight, with respect to the weight of the composition.

Volatile Oil

According to an advantageous embodiment of the invention, the composition contains no (0%) or very little (maximum 5% by weight with respect to the total weight of the composition) volatile oil.

“Volatile oil” denotes an oil suitable for evaporating in contact with skin in less than one hour, at ambient temperature and atmospheric pressure.

The volatile oil is a volatile cosmetic oil, liquid at ambient temperature, having particularly a non-zero vapour pressure at ambient temperature and atmospheric pressure, in particular having a vapour pressure of between 0.13 Pa and 40,000 Pa (0.001 to 300 mm of Hg), preferably between 1.3 Pa and 13,000 Pa (0.01 to 100 mm of Hg), and more preferentially between 1.3 Pa and 1,300 Pa (0.01 to 1,000 mm of Hg).

The volatile oils comprise volatiles silicone oils and/or volatile hydrocarbon oil.

The volatile silicone oils optionally used in the compositions according to the invention are linear or cyclic, have particularly from 2 to 7 silicon atoms, optionally alkyl or alkoxy groups having from 1 to 10 carbon atoms, and having a viscosity, at ambient temperature, less than 5 cSt.

By way of examples of volatile silicone oil, mention may more particularly be made of hexamethylcyclotrisiloxane, octamethylcyclotetrasiloxane, decamethylcyclopentasiloxane, dodecamethylcyclohexasiloxane, cyclotetradimethylsiloxane, cyclopentadimethylsiloxane, cyclohexadimethylsiloxane, hexamethyldisiloxane, octamethyltrisiloxane, hexylheptamethyltrisiloxane, octylheptamethyltrisiloxane, decamethyltetrasiloxane, dodecamethylpentasiloxane, heptamethylhexyl trisiloxane, heptamethyloctyl trisiloxane or one of the mixtures thereof.

Concerning the volatile hydrocarbon oil, mention may more particularly be made of a short-chain hydrocarbon oil, volatile linear alkanes such as for example described in document FR2933865 incorporated by way of reference.

By way of examples of short-chain hydrocarbon oil(s), mention may particularly be made of that/those chosen in the group comprising isododecane, isodecane, isohexadecane, dodecane or one of the mixtures thereof.

By way of example of volatile linear alkanes, mention may be made of those hydrocarbon chains in:
C9-C17, C10-C14, such as a mixture of undecane and tridecane, marketed by BASF Care Creations under the trade name Cetiol® Ultimate,
C15-19, such as those marketed by Seppic under the trade name Emogreen L15,
C12-14, such as those marketed by Biosynthis under the trade name Vegelight 1214LC.
C9-12 alkane, such as those marketed by Daito under the trade name Makigreen D10.

### The Structuring Agent

The structuring agent of the oily phase comprises at least one wax and/or at least one silicone resin and/or a lipophilic gelling agent.

The term “wax” denotes a liquid/solid reversible change fat, having a melting point greater than 30° C. and generally less than 110° C., which is liquid under the composition preparation conditions and which has an anisotropic crystalline organisation in the solid state.

The term “lipophilic gelling agent” denotes a substance capable of solidifying or gelling the oil introduced into the composition according to the invention.

According to an embodiment of the invention, the structuring agent is a mixture of at least one wax and at least one lipophilic gelling agent.

According to a further embodiment of the invention, the structuring agent is a mixture of at least one wax and at least one silicone resin.

According to a further embodiment of the invention, the structuring agent is a mixture of at least two waxes.

The wax suitable for the cosmetic compositions according to the invention comprises at least one polar wax and/or at least one apolar wax.

Polar wax denotes a wax comprising at least one heteroatom such as oxygen, nitrogen, silicon or phosphorus.

In particular, the polar wax may be chosen in the group comprising beeswax, carnauba wax, candelilla wax, cotton wax, rice bran wax, berry wax, Chinese insect wax, montan wax, lanolin and the alcohol, acetylated, esterified, polyethoxylated derivatives thereof, kapok wax, sugarcane wax, hexyl laurate, jojoba wax, shellac wax, polyethoxylated cholesterol ether, synthetic beeswaxes marketed by Koster Keunen under the trade name Kester Wax K82H, or one of the mixtures thereof.

Mention may also be made of plant ester waxes chosen in the group comprising the mixture of esters of jojoba, polyglycerin-3, Acacia decurrens flower wax and sunflower seed wax, said mixture being marketed by Gattefosse under the trade name Acticire®, jojoba esters marketed by Floratech under the trade name Floraesters 60 or Floraesters 70, alkyl esters of hydrogenated alkyl esters marketed by Sophim under the trade name Phytowax, such as for example hydrogenated lauroyl oleate esters marketed under the trade name Phytowax Olive 12L44.

Apolar wax denotes a hydrocarbon wax and/or a silicone wax.

“Hydrocarbon apolar wax” denotes a wax only comprising carbon and hydrogen atoms and comprising no heteroatoms such as oxygen, nitrogen, silicon or phosphorus.

Examples of suitable hydrocarbon apolar waxes in the compositions according to the invention comprise polyethylene wax, marketed by New Phase Technologies under the trade name Performalene 400 (P400) or by Jeen International Corporation under the trade name Jeenate 3H, a mixture of high-molecular-weight linear polyethylene and ethylene/propylene copolymer, marketed by Safic-Alcan under the trade name Lipwax® PZ80-20, a synthetic wax marketed by Sasol under the trade name Sasol Wax C80, synthetic waxes and plant waxes, such as for example a mixture of synthetic wax and carnauba (Copernicia cerifera) wax marketed by Strahl & Pitsch under the trade name Smart wax 202, a mixture of synthetic wax, Candelilla wax and carnauba (Copernicia cerifera) wax marketed by Strahl & Pitsch under the trade name Smartwax 7743S, Fischer Tropsch waxes marketed by Cirebelle under the trade name Cirebelle 303, or one of the mixtures thereof.

“Silicone apolar wax” denotes a wax comprising a silicon heteroatom.

Examples of suitable silicone apolar waxes in the compositions according to the invention comprise C20-24 alkyl dimethicone, marketed by Siltech under the trade name Silwax D2024, C24-28 alkyl dimethicone, marketed by Evonik Industries AG under the trade name Abil Wax, or one of the mixtures thereof.

More particularly, according to a further embodiment of the invention, the wax is chosen in the group comprising beeswax, polyethylene wax, marketed by New Phase Technologies under the trade name Performalene 400 (P400) or by Jeen International Corporation under the trade name Jeenate 3H, a mixture of high-molecular-weight linear polyethylene and ethylene/propylene copolymer, marketed by Safic-Alcan under the trade name Lipwax® PZ80-20, a synthetic wax marketed by Sasol under the trade name Sasol Wax C80, a mixture of high-molecular-weight linear polyethylene and ethylene/propylene copolymer, marketed by Safic-Alcan under the trade name Lipwax® PZ80-20.

Thus according to a preferred embodiment of the invention, the structuring agent is a wax chosen from among beeswax, synthetic wax, polyethylene wax or one of the mixtures thereof.

According to an embodiment of the invention, the wax is present in the cosmetic composition according to the invention at a content of between 10% and 30%, preferably between 15% and 25%, the percentages being percentages by weight with respect to the total weight of the composition.

By way of example of silicone resins, mention may be made of:
siloxysilicates which may be trimethylsiloxysilicate of formula [(CH3)3SiO]x(SiO4/2)y (MQ units) wherein x and y are integers between 50 and 80,
polysilesquioxanes of formula (CH3SiO3/2).x (T units) wherein x is greater than 100 and of which at least one of the methyl radicals may be substituted by a group R as defined above, polymethylsilsesquioxanes which are polysilsesquioxanes wherein none of the methyl radicals is substituted by another group. Such polymethylsilsesquioxanes are described in the document U.S. Pat. No. 5,246,694.

By way of examples of commercially available polymethylsilsesquioxane resins, mention may be made of those marketed:
by the company Wacker under the reference Resin MK such as Belsil PMS MK: polymer comprising repeat CH3SiO3/2 units (T units), optionally also comprising up to 1% by weight of (CH3)2SiO2/2 units (D units) and having a mean molecular weight of about 10,000,
by the company SHIN-ETSU under the references KR-220L which are composed of T units of formula CH3SiO3/2 and have terminal Si—OH (silanol) groups, under the reference KR-242A which comprise 98% T units and 2% D dimethyl units and have terminal Si—OH groups or indeed under the reference KR-251 comprising 88% T units and 12% D dimethyl units and have terminal Si—OH groups.

As siloxysilicate resins, mention may be made of trimethylsiloxysilicate (TMS) resins optionally in powder form. Such resins are marketed under the reference SR1000 by the company General Electric or under the reference TMS 803 by the company Wacker. Mention may further be made of trimethylsiloxysilicate resins marketed in a solvent such as cyclomethicone, sold under the trade name “KF-7312J” by the company Shin-Etsu, “DC 749”, “DC 593” by the company Dow Corning.

The lipophilic gelling agents suitable for being used in the compositions according to the invention may be organic or mineral, polymeric or molecular lipophilic gelling agents.

As mineral lipophilic gelling agent, mention may be made of optionally hydrophobically surface-treated pyrogenic silica the size whereof is less than 1 μm. It is indeed possible to chemically modify the surface of the silica, by chemical reaction generating a decrease in the number of silanol groups present on the silica surface. Silanol groups may particularly be substituted by hydrophobic groups: a hydrophobic silica is then obtained. The hydrophobic groups may be:
trimethylsiloxyl groups, which are particularly obtained by treating pyrogenic silica in the presence of hexamethyldisilazane. Silicas treated in this way are known as “Silica silylate” as per the CTFA (8^{th} edition, 2000). They are for example marketed under the references Aerosil R812® by the company DEGUSSA, CAB-O-SIL TS-530® by the company CABOT,
dimethylsilyloxyl or polydimethylsiloxane groups, which are particularly obtained by treating pyrogenic silica in the presence of polydimethylsiloxane or dimethyldichlorosilane. Silicas treated in this way are known as “Silica dimethyl silylate” as per the CTFA (8^{th} edition, 2000). They are for example marketed under the references Aerosil R972®, and Aerosil R974® by the company DEGUSSA, CAB-O-SIL TS-610® and CAB-O-SIL TS-720® by the company CABOT.

The hydrophobic pyrogenic silica has in particular a particle size that may be nanometric to micrometric, for example ranging from about 5 to 200 nm.

The polymeric organic lipophilic gelling agents are for example partially or totally crosslinked elastomeric organopolysiloxanes, of three-dimensional structure, such as those marketed under the trade names of KSG6®, KSG16® and KSG18® by the company SHIN-ETSU, Trefil E-505C® and Trefil E-506C® by the company DOW-CORNING, Gransil SR-CYC®, SR DMF10®, SR-DC556®, SR 5CYC Gel®, SR DMF 10 Gel® and SR DC 556 Gel® by the company GRANT INDUSTRIES, SF 1204® and JK 113® by the company GENERAL ELECTRIC; ethylcellulose such as that sold under the trade name Ethocel® by the company DOW CHEMICAL; galactomannans including from one to six, and in particular from two to four, hydroxyl groups per monosaccharide, substituted by an optionally saturated alkyl chain, such as guar gum alkylated by C1 to C6, and in particular C1 to C3, alkyl chains or one of the mixtures thereof. “Diblock”, “triblock” or “radial” sequenced copolymers of the polystyrene/polyisoprene, polystyrene/polybutadiene type such as those marketed under the trade name Luvitol HSB® by the company BASF, of the polystyrene/copoly(ethylene-propylene) type such as those marketed under the trade name of Kraton® by the company SHELL CHEMICAL CO or indeed of the polystyrene/copoly(ethylene-butylene) type, mixtures of triblock and radial (star) copolymers in isododecane such as those marketed by the company PENRECO under the trade name Versagel® such as for example the mixture of butylene/ethylene/styrene triblock copolymer and ethylene/propylene/styrene copolymer in isododecane (Versagel M 5960) or in hydrogenated polyisobutene (Versagel ME 2000).

A further type of polymeric organic lipophilic gelling agent consists of polyamide resins or poly (ester-amide) resins, such as ester-terminated polyamides (ETPA), ester-terminated poly (ester-amides) (ETPEA), tertiary amide-terminated polyamides (ATPA), polyalkyleneoxy-terminated polyamides (PAOPA) or polyether polyamides (PEPA).

Examples of ester-terminated polyamides (ETPA) are those identified by the INCI name “Ethylenediamine/Stearyl Dimer Dilinoleate Copolymer” and available, for example, under the trade name Uniclear® 100VG from the company Arizona Chemical.

Examples of ester-terminated poly (ester-amides) (ETPEA) are those identified by the INCI name polyamide-8 which are “Bis-stearyl ethylenediamine/neopentylglycol/stearyl ethylenediamine dibenzoate dimer copolymers” and available, for example, under the trade name Oloecraft® LP-20-PA-MV from the company Croda.

Examples of tertiary amide-terminated polyamides (ATPA) are those identified by the INCI name “Ethylenediamine/Hydrogenated Dimer Dilinoleate Copolymer Bis-Di-C14-18 Alkyl Amine” and available, for example, under the trade name Sylvaclear® A200V or Sylvaclear® A2614V from the company Arizona Chemical or those identified by the INCI name “diisostearyl malate and ethylenediamine/hydrogenated dimer dilinoleate copolymer bis-di-c14-18 alkyl amide” and available, for example, under the trade name Haimalate PAM from the company Kokyu Alcohol Kogyo.

Examples of polyalkyleneoxy-terminated polyamides (PAOPA) are those identified by the INCI name Polyamide-3 and available, for example, under the trade name Sylvaclear® AF1900V, Sylvaclear® PE1800V and Sylvaclear® PA1200V from the company Arizona Chemical.

Examples of polyether polyamides (PEPA) are those identified by the INCI name Polyamide-6 and available, for example, under the trade name Sylvaclear® PE400V from the company Arizona Chemical.

A further type of polymeric organic lipophilic gelling agent consists of N-acyl glutamic acid diamides. Mention may particularly be made of an N-acyl glutamic acid diamide having a straight-chain alkyl group such as dibutyl lauroyl glutamide and an N-acyl glutamic acid diamide having a branched-chain alkyl group, such as dibutyl ethylhexanoyl glutamide. Dibutyl lauroyl glutamide is commercially available as GP-1 and dibutyl ethylhexanoyl glutamide is commercially available under the name EB-21, and are both marketed by Ajinomoto.

A further type of polymeric organic lipophilic gelling agent consists of dextrin esters. Mention may be made of esters of dextrin and fatty acids, such as dextrin palmitate.

A further type of polymeric organic lipophilic gelling agent consists of sucrose esters. mention may be made of esters of sucrose and fatty acids, such as sucrose tetrastearate triacetate available under the trade name Sisterna® A10E-C from the company Sisterna.

A further type of polymeric organic lipophilic gelling agent consists of glyceryl esters. Mention may be made of eicosadioic acid diester and glycerol esterified with behenic acid. It is in particular available under the trade name NOMCORT® HK-G from the company NISSHIN OILLIO.

Advantageously, the composition according to the invention may comprise from 0.5 to 40% by weight of structuring agents other than waxes with respect to the total weight of the composition, preferably from 1 to 30% or more preferably, from 5 to 30%.

### Pasty Compounds

The composition according to the invention may further comprise a pasty compound which may be advantageously chosen from among:
lanolin and derivatives thereof
optionally polymeric silicone compounds
optionally polymeric fluorinated compounds
vinyl polymers, in particular:
   olefin homopolymers
   olefin copolymers
   hydrogenated diene homopolymers and copolymers
   linear or branched alkyl (meth)acrylate homo or copolymer oligomers having preferably a C8-C30 alkyl group
   vinyl ester homo and copolymer oligomers having C8-C30 alkyl groups
   vinyl ether homo and copolymer oligomers having C8-C30 alkyl groups,
liposoluble polyethers resulting from polyetherification between one or a plurality of C2-C100, preferably C2-050, diols,
beeswax and octyldodecanol mixtures such as that marketed under the trade name Zenibee Cream by the company Zenitech,
esters,
plant butters such as mango, shea, cocoa, cotton, avocado butters, etc.
   or one of the mixtures thereof.

Among esters, the following may particularly be used:
oligomeric glycerol esters, particularly diglycerol esters, in particular condensates of adipic acid and glycerol, for which a portion of the hydroxyl groups of the glycerols have reacted with a mixture of fatty acids such as stearic acid, capric acid, stearic acid and isostearic acid and 12-hydroxystearic acid, like particularly those marketed under the brand Softisan 649 by the company Sasol,
arachidyl propionate marketed under the brand Waxenol 801 by Alzo,
phytosterol esters such as the product under the INCI name
“Phytosteryl/behenyl/octyldodecyl/isostearyl lauroyl glutamate” marketed under the trade name of Eldew-PS308 by the company Ajinomoto,
fatty acid triglycerides and derivatives thereof, for example the stearyl heptanoate and stearyl caprylate mixture marketed under the trade name DUB sold by the company Stéarinerie Dubois,
pentaerythritol esters,
non-crosslinked polyesters resulting from polycondensation between a dicarboxylic acid and a linear or branched C4-C50 carboxylic polyacid and a diol or a C2-C50 polyol,
ester aliphatic esters resulting from esterification of an aliphatic hydroxycarboxylic acid ester by an aliphatic carboxylic acid such as cetyl lactate marketed under the trade name ceraphyl 28 by the company ISP (International Speciality Products),
polyesters resulting from esterification, by a polycarboxylic acid, of an aliphatic hydroxy carboxylic acid ester, said ester comprising at least two hydroxyl groups such as the products Risocast DA-H®, and Risocast DA-L®,

or one of the mixtures thereof.

Among the pasty compounds, the following will preferably be chosen:
optionally polymeric silicone compounds

or a mixture thereof.

The composition according to the invention may comprise a total content of pasty compounds of between 1 and 40% by weight with respect to the total weight of the composition, in particular between 4% and 30%, more particularly between 5% and 20%.

### Additional Fillers

The lipstick composition may also contain further fillers.

These filers are preferably colourless or white. 1

The constituent particles thereof may be optionally porous, and be presented in various shapes, particularly in sheet, spherical or oblong shape, regardless of the crystallographic shape (for example laminate, cubic, hexagonal, orthorhombic, etc).

They are chosen so as not to disrupt the sought result, therefore, not to absorb the oils and not to add gloss.

In particular, the additional filler may be chosen from among lauroyl lysine, boron nitride, silicone microbeads such as those marketed under the trade name Tospearl by Toshiba for example, precipitated calcium carbonate, hydroxyapatite, elastomeric polyorganosiloxane particles, glass or ceramic microcapsules, zinc laurate, magnesium myristate, magnesium and aluminium silicate such as that marketed under the trade name Neusilin ULF2 by the company Fuji Chemical Industry, starch, a clay or one of the mixtures thereof.

Among the additional fillers, starch, a clay or one of the mixtures thereof are preferred.

The starch may be chosen for example from a rice, tapioca, potato or corn starch. Rice starch is preferred particularly that of INCI name distarch phosphate marketed under the name “Rice PO4 Natural” by the company Agrana Starch. It absorbs very little of the oils and has a matte appearance and adds softness to the composition according to the invention which prevents the grating aspect of the particular filler as mattifying agent.

Starch may be present in the lipstick composition in a quantity of between 2.0% and 10.0% by weight, preferably between 6.0 and 10.0% by weight with respect to the total weight of the composition.

The clay may be natural or synthetic. It is rendered lipophilic with a treatment with an alkyl ammonium salt such as a C10 to C22 ammonium chloride, for example di-stearyl dimethyl ammonium chloride. It may be chosen from among bentonites in particular hectorites and montmorillonites, beidellites, saponites, nontronites, sepiolites, biotites, attapulgites, and vermiculites. Preferably, the clay is chosen from among hectorites. By way of example of hectorite, mention may be made of the product sold under the Bentone 38V CG by the company ELEMENTIS SPECIALTIES (INCI name disteardimonium hectorite).

It has the advantage of increasing the mattifying effect while acting as a rheology modifier. It may be present in a proportion of between 0.1% and 4% and preferably between 0.5% and 2% by weight with respect to the total weight of the composition.

### Additional Additives

Besides the constituents cited above, the composition according to the invention may contain various ingredients, such as a colouring agent, a UV filter, or one of the mixtures thereof.

The colouring agent may particularly be chosen from among water-soluble or liposoluble colorants, pigments, nacres, lacquers or one of the mixtures thereof. These colouring agents may be optionally surface-treated with a hydrophobic agent such as silanes, silicones, fatty acid soaps, C₉₋₁₅ fluoroalcohol phosphates, acrylate/dimethicone copolymers, mixed C₉₋₁₅ fluoroalcohol phosphate/silicone copolymers, lecithins, carnauba wax, polyethylene, chitosan and optionally acylated amino acids such as lauroyl lysine, disodium stearoyl glutamate and aluminium acyl glutamate. The pigments may be mineral or organic, natural or synthetic.

Examples of mineral pigments are particularly titanium dioxide, iron, zinc or chromium oxides, manganese violets, ultramarines, ferric ferrocyanide known as Prussian Blue, as well as composite pigments and goniochromatic, pearlescent, interferential, photochromic or thermochromic pigments, without this list being exhaustive.

Examples of organic pigments suitable for use in the invention are particularly carbon black, D&C type pigments, lacquers based on cochineal carmine, barium, strontium, calcium or aluminium or indeed diketopyrrolopyrrole (DPP) described in the documents EP-A-542669, EP-A-787730, EP-A-787731 and WO-A-96/08537.

The nacres may be chosen from among those conventionally present in makeup products, such as micas/titanium dioxide. Alternatively, they may consist of nacres based on mica/silica/titanium dioxide, based on synthetic fluorphlogopite/titanium dioxide (MAPRECOS SUNSHINE®), calcium sodium borosilicate/titanium dioxide (ENGELHARD REFLECKS®) or calcium aluminium borosilicate/silica/titanium dioxide (MERCK RONASTAR®).

Advantageously, when it contains one or a plurality of pigments, the composition according to the invention further contains at least one dispersant such as diisostearyl malate.

The colouring agents are present in the composition at a content of between 5% and 8%, the percentages being percentages by weight with respect to the total weight of the composition.

The UV filters may particularly be chosen from among organic and inorganic filters or one of the mixtures thereof. As organic filters, mention may be made particularly of dibenzoylmethane derivatives (of which butyl methoxydibenzoylmethane), cinnamic acid derivatives (of which ethylhexyl methoxycinnamate), salicylates, para-aminobenzoic acids, β,β′-diphenylacrylates, benzophenones, benzylidene camphor derivatives, phenylbenzimidazoles, triazines, phenylbenzotriazoles and anthranilic derivatives. As inorganic filters, mention may particularly be made of filters based on mineral oxides in the form of pigments or nanopigments, optionally coated, and in particular based on titanium dioxide or zinc oxide.

The composition according to the invention may also contain plant butters or butters of synthetic origin. In any case, these are chosen to avoid adding gloss. The use of butters of matte appearance such as mango butter for example is preferred.

The composition according to the invention may also contain one or a plurality of sweetening agents such as sorbitol, sucrose, xylitol, acesulfame K and sodium saccharinate; antioxidants such as ascorbic acid alkyl or phosphoryl esters, or indeed tocopherol and the esters thereof; sequestering agents such as EDTA salts; pH adjusters; preservatives; fragrances; vitamins; hydrating agents; or one of the mixtures thereof.

Examples of such adjuvants are cited in particular in the CTFA Dictionary (International Cosmetic Ingredient Dictionary and Handbook published by The Cosmetic, Toiletry and Fragrance Association, 11^{th} Edition, 2006).

### Matte Appearance of the Composition

The lipstick composition has advantageously a matte appearance and a homogeneous and creamy texture.

The matte appearance of the lipstick composition is demonstrated by a gloss measurement.

The lipstick composition according to the invention spread on a contrast chart with a white background over a thickness of 500 microns thanks to an automatic spreader has a gloss value measured at 85° using a glossmeter named micro-TRI-gloss or PICOGLOSS 503 sold by the company BYK of less than 30, preferably less than 20, more preferably less than 10.

The gloss value is obtained with the means of 3 measurements made at three different points of the contrast chart.

### Hardness of the Composition

One of the advantages of the composition according to the invention is that the texture thereof is homogeneous and creamy and that it remains so over time.

This property of textural stability over time is measured thanks to a hardness measured.

The cosmetic composition according to the invention as defined above may further be characterised in that it has a hardness of less than 300 g (grams), 1 day after manufacture and after storage of 1 day, 15 days, 1 month and 2 months in an oven at 45° C.

The hardness of the composition, which is expressed in grams (g), is determined by measuring the compression force measured at 20° C. using a texture analyser sold under the trade name “TA-XT Plus Microstable System” by the company Swantech. The texture analyser is equipped with a stainless steel cylinder of a diameter 2 mm moving at the measurement speed of 1 mm/s and penetrating the composition at a depth of 3 mm.

The hardness value is the compression force measured, divided by the surface area of the texture analyser cylinder in contact with the composition. The samples are poured hot and to the top into a round 60 mm radius and 15 mm high Petri dish. The samples prepared are stored for 24 h to 48 h at 20° C. before performing measurement.

The present invention also relates to a lipstick comprising:
From 8% to 20% by weight of mattifying filler with respect to the total weight of the composition
From 5% to 8% by weight of colouring agent with respect to the total weight of the composition
From 10% to 30% and preferably from 15% to 25% by weight of structuring agent with respect to the total weight of the composition
From 40% to 80%, and preferably from 45% to 70% and more preferably from 50% to 60% of oil with respect to the total weight of the composition.

### Method

The present invention also relates to providing a method for preparing a matte lipstick composition. This other aspect of the invention is characterised in that a matte lipstick composition is prepared by mixing at least one particular filler as a mattifying agent associated with at least one oil and at least one structuring agent. A lipstick composition obtained according to the method of the invention advantageously has a matte appearance and a homogeneous and creamy texture.

The present invention also relates to providing a method for preparing a matte lipstick composition comprising the following steps:
(1) Grinding the pigments, if present, beforehand in a portion of the oil;
(2) Melting the structuring agent and the rest of the oil;
(3) Adding to the molten mixture obtained in (2), the particular filler as a mattifying agent, any colouring agents other than the pigments and any additional fillers as well as any pigments from step (1) under continuous stirring;
(4) Optionally adding the active substances or fragrances; and
(5) Pouring the hot composition obtained in step (4) into moulds, then allowing to cool until solidification.

The gloss and hardness measurements are then carried out.

The following examples are given to illustrate the invention. These examples merely being given by way of illustration, the invention may in no way be restricted to the subject matter thereof.

### EXAMPLE I: LIPSTICK PREPARATION

Lipstick compositions of the following formulas (% by weight) are prepared:

TABLE 1

| | | | invention | | | | comparative | | |
|---|---|---|---|---|---|---|---|---|---|
| | | | Ex 1 | Ex 2 | Ex 3 | Ex 4 | Ex 5 | Ex 6 | Ex 7 |
| function | Ingredient | TRADE NAME | (%) | (%) | (%) | (%) | (%) | (%) | (%) |
| wax | Ethylene/propylene | LIPWAX PZ80-20 | 11.10 | 11.10 | 11.10 | 11.10 | 11.10 | 11.10 | 11.10 |
| | copolymer and | | | | | | | | |
| | synthetic wax | | | | | | | | |
| wax | Hydrogenatedcastor oil | CASTORWAX MP 80 | 3.46 | 3.46 | 3.46 | 3.46 | 3.46 | 3.46 | 3.46 |
| wax | Natural beeswax | CERABEIL BLANCHE | 5.00 | 5.00 | 5.00 | 5.00 | 5.00 | 5.00 | 5.00 |
| oil | Isononyl isononanoate | DUB ININ | 17.54 | 17.54 | 17.54 | 17.54 | 17.54 | 17.54 | 17.54 |
| oil | Diisostrearyl malate | SALACOS 222 | 17.68 | 17.68 | 17.68 | 17.68 | 17.68 | 17.68 | 17.68 |
| oil | Dicaprilyl carbonate | CETIOL CC | 12.8 | 12.8 | 12.8 | 12.8 | 12.8 | 12.8 | 12.8 |
| thickener | Dicaprilyl carbonale | COSMEDIA GEL CC | 9.82 | 9.82 | 9.82 | 9.82 | 9.82 | 9.82 | 9.82 |
| | (8.56%) and stearalkonium | | | | | | | | |
| | hectorite (0.98%) and | | | | | | | | |
| | propylene carbonate (0.29%) | | | | | | | | |
| active substance | Tocopheryl acetate | DL-ALPHA- | 0.5 | 0.5 | 0.5 | 0.5 | 0.5 | 0.5 | 0.5 |
| | | TOCOPHERYL ACETATE | | | | | | | |
| Additional | Magnesium aluminium silicate | NEUSILIN ULF2 | 1.00 | 1.00 | 1.00 | 1.00 | 1.00 | 1.00 | 1.00 |
| filler | | | | | | | | | |
| Colouring agent | Pigments | | 7.00 | 7.00 | 7.00 | 7.00 | 7.00 | 7.00 | 7.00 |
| fragrance | Parfum | | 0.08 | 0.08 | 0.08 | 0.08 | 0.08 | 0.08 | 0.08 |
| filler according | Basic magnesium carbonate | ICL Industrial products | 14.0 | — | — | — | — | — | — |
| to the invention | | Basic magnesium carbonate | | | | | | | |
| filler according | Triethoxycaprylilsilane-treated | Triethoxycaprylilsilane- | — | 14.0 | — | — | — | — | — |
| to the invention | basic magnesium carbonate | treated ICL Industrial | | | | | | | |
| | | products Basic magnesium | | | | | | | |
| | | carbonate | | | | | | | |
| filler according | Diatomaceous earth | IMERYS IMERCARE ® | — | — | 14.0 | — | — | — | — |
| to the invention | | 03D | | | | | | | |
| filler according | Microcrystalline cellulose | VIVAPUR ®CS 4FM | — | — | — | 14.0 | — | — | — |
| to the invention | | | | | | | | | |
| non-inventive | PE magnesium carbonate | SCORA magnesium | — | — | — | — | 14.0 | — | — |
| filler | sheet | carbonate sheet | | | | | | | |
| non-inventive | Tubular magnesium | Nittetsu Mimming | — | — | — | — | — | 14.0 | — |
| filler | carbonate | MgTube ® | | | | | | | |
| non-inventive | Methyl methacrylate | TECHPOLYMER MBP-8 | — | — | — | — | — | — | 14.0 |
| filler | crosspolymer | | | | | | | | |
| Total | | Total composition: | 100 | 100 | 100 | 100 | 100 | 100 | 100 |

### Procedure:

The pigments are ground beforehand in a portion of the oily ingredients. Then, the polymer or wax structuring ingredients and the remaining oily ingredients are melted separately and the mattifying agents and additional filers are added followed by the ground pigments to the preceding mixture under continuous stirring. Finally, suitable active substances or fragrances may be added. Hot pouring is then performed into moulds. Mould release is then performed after cooling.

### EXAMPLE II: SPECIFIC SURFACE AREA MEASUREMENTS OF MATTIFYING FILLERS

The specific surface area measurements of the mattifying fillers are carried out using a BET tristar II measuring apparatus. The method is based on the adsorption of nitrogen molecules on the surface and in the pores of the powder sample at low temperature (nitrogen liquefaction temperature: 77° K). The specific surface area is expressed in m²/g.

TABLE 2

| | | | | | specific |
|---|---|---|---|---|---|
| | | | | particle | surface |
| example | Trade name | INCI name | supplier | size | area (m²/g) |
| Inventive | Basic magnesium | MAGNESIUM | ICL | from 5 to | 4.1 |
| filler Ex1 | carbonate | CARBONATE | Industrial | 25 μm | |
| | | | products | | |
| Inventive | Triethoxycaprylyl- | MAGNESIUM | ICL | 5 to 20μm | 4.7 |
| filler Ex2 | silane-treated basic | CARBONATE | Industrial | | |
| | magnesium | | products | | |
| | carbonate | | | | |
| Inventive | Imercare ® 03D | DIATOMACEOUS | IMERYS | 2 to 5 μm | 12 |
| filler Ex3 | | EARTH | | | |
| Inventive | VIVAPUR ®CS | MICROCRYSTALLINE | RETTENMAIER | 0.9 0 | 5.7 |
| filler Ex4 | 4FM | CELLULOSE | | 9.4 μm | |
| Non- | MgC03 PE sheet | MAGNESIUM | SCORA | 3 to 25 μm | 30.4 |
| inventive | | CARBONATE | | | |
| filler Ex5 | | | | | |
| Non- | MgTube ® | TUBULAR | Nittetsu | tube 20 to | 107.5 |
| inventive | | MAGNESIUM | Mimming | 60 μm | |
| filler Ex6 | | CARBONATE | | ovoid 1 to | |
| | | | | 15 μm | |
| Non- | TECHPOLYMER | METHYL | Sekuisi | 4.8 to 14 | 81.9 |
| inventive | MBP-8 | METHACRYLATE | Plastics co | microns | |
| filler Ex7 | | CROSSPOLYMER | Ltd | | |

### EXAMPLE III— GLOSS AND HARDNESS MEASUREMENTS

### Gloss Measurement

The lipstick composition from each example prepared in example I is spread on a contrast chart with a white background over a thickness of 500 microns thanks to an automatic spreader has a gloss value measured at 85° using a glossmeter named micro-TRI-gloss or PICOGLOSS 503 sold by the company BYK.

The gloss value is obtained with the means of 3 measurements made at three different points of the contrast chart and given in table 3.

### Hardness Measurement

The hardness of the composition, which is expressed in grams (g), is determined by measuring the compression force measured at 20° C. using a texture analyser sold under the trade name “TA-XT Plus Microstable System” by the company Swantech. The texture analyser is equipped with a stainless steel cylinder of a diameter 2 mm moving at the measurement speed of 1 mm/s and penetrating the composition at a depth of 3 mm.

The hardness value is the compression force measured, divided by the surface area of the texture analyser cylinder in contact with the composition.

The samples of the lipstick compositions from each example prepared in example I are poured hot and to the top into a round 60 mm radius and 15 mm high Petri dish. The samples prepared are stored for 24 h to 48 h at 20° C. before performing measurement. The results are given in table 3.

TABLE 3

| | | Gloss at | Hardness | Hardness | Hardness | Hardness |
|---|---|---|---|---|---|---|
| Trade name | | 85° with | (g) | (g) | (g) D + 1 | (g) D + 2 |
| Mattifying filler | Example | BYK | D + 1 | D + 15 | month | months |
| basic magnesium | Inventive | 7.3 +/− 0.5 | 280 +/− 4 | 267 +/− 6 | 270 +/− 3 | 257 +/− 4 |
| carbonate | Example 1 | | | | | |
| Triethoxyxaprylylsilane- | Inventive | 8.1 +/− 0.4 | 230 +/− 8 | 226 +/− 10 | 227 +/− 7 | 215 +/− 9 |
| treated magnesium | Example 2 | | | | | |
| carbonate | | | | | | |
| Imercare ® 03D | Inventive | 0.4 +/− 0.1 | 223 +/− 4 | 215 +/− 12 | 217 +/− 13 | 214 +/− 6 |
| diatomaceous earth | Example 3 | | | | | |
| VIVAPUR ®CS 4FM | Inventive | 2.7 +/− 0.5 | 245 +/− 4 | 244 +/− 8 | 246 +/− 3 | 262 +/− 3 |
| | Example 4 | | | | | |
| MgC03 PE sheet | Comparative | 1.1 +/− 0.2 | 400 +/− 3 | 400 +/− 5 | 410 +/− 5 | 403 +/− 4 |
| | Example 5 | | | | | |
| MgTube ® | Comparative | 1 +/− 0.1 | 457 +/− 10 | 512 +/− 4 | 463 +/− 9 | 484 +/− 5 |
| | Example 6 | | | | | |
| TECHPOLYMER MBP-8 | Comparative | 2.5 +/− 0.6 | 322 +/− 4 | 321 +/− 5 | 336 +/− 5 | 315 +/− 4 |
| | Example 7 | | | | | |

The compositions of example 1 and example 2 with a magnesium carbonate of specific surface area meeting the criteria of the invention have a homogeneous and creamy texture characterised by the hardness thereof which remains stable at a value less than 300 g (grams) over time even after storage for 15 days, 1 month and 2 months at 45° C.

The compositions of example 3 with a diatomaceous earth of specific surface area meeting the criteria of the invention have a homogeneous and creamy texture characterised by the hardness thereof which remains stable at a value less than 300 g (grams) over time even after storage for 15 days, 1 month and 2 months at 45° C.

The compositions of example 4 with a microcrystalline cellulose of specific surface area meeting the criteria of the invention have a homogeneous and creamy texture characterised by the hardness thereof which remains stable at a value less than 300 g (grams) over time even after storage for 15 days, 1 month and 2 months at 45° C.

On the other hand, the compositions of comparative examples 5 and 6 with a magnesium carbonate of specific surface area not meeting the criteria of the invention have a much harder texture from manufacture, with a value greater than 400 g (grams) and which increases further in hardness over time after storage for 15 days, 1 month and 2 months at 45° C.

The composition of comparative example 7 with a methyl methacrylic crosspolymer (Techpolymer MBP-8) of specific surface area not meeting the criteria of the invention has a much harder texture from manufacture with a value greater than 300 g (grams) and which increases further in hardness over time after storage for 15 days, 1 month and 2 months at 45° C.

1-19. (canceled)

20. A matte lipstick composition comprising at least one structuring agent, at least one oil, at least one colouring agent, and at least one particular filler as a mattifying agent, wherein the particular filler serving as a mattifying agent is selected from among particles having a specific surface area of less than 30 m²/g, preferably less than 20 m²/g, more preferably still less than 10 m²/g and even more advantageously less than 5 m²/g.

21. The matte lipstick composition according to claim 20, wherein the particular filler is chosen from among magnesium carbonate powders, in particular basic magnesium carbonate of formula (MgCO₃)₃.Mg(OH)₂.3H₂O or of formula (MgCO₃)₄.Mg(OH)₂.5H₂O or indeed normal magnesium carbonate of formula (MgCO₃).xH₂O, among diatomaceous earths, and among microcrystalline cellulose, or one of the mixtures thereof.

22. A matte lipstick composition comprising at least one structuring agent, at least one oil, at least one colouring agent, and at least one particular filler as a mattifying agent, wherein the particular filler serving as a mattifying agent is selected from among magnesium carbonate powders having a specific surface area of less than 10 m²/g and even more advantageously less than 5 m²/g.

23. The matte lipstick composition according to claim 20, wherein the total content of particular filler serving as a mattifying agent in the composition is between 8% and 20% and preferably between 10% and 15% by weight with respect to the total weight of the composition.

24. The matte lipstick composition according to claim 20, wherein the oil is chosen from among silicone or hydrocarbon non-volatile oils or one of the mixtures thereof.

25. The matte lipstick composition according to claim 20, wherein the composition comprises less than 5% volatile oil and preferably is free from volatile oil.

26. The matte lipstick composition according to claim 24, wherein the non-volatile oil is chosen from among synthetic (poly) esters and (poly) ethers, in particular (poly) esters of C6-C20 acids and C6-C20 alcohols, advantageously branched, such as isononyl isononanoate; di(C6-C20 alkyl) carbonates such as dicaprylyl carbonate marketed by BASF under the trade name Cetiol CC; branched and/or unsaturated fatty acids; polyesters of polyols, in particular of (di) pentaerythritol, such as pentaerythritol tetraoctanoate, silicone oils such as linear polydimethylsiloxanes of viscosity between 5 cSt and 100 cSt (centistokes); or one of the mixtures thereof.

27. The matte lipstick composition according to claim 24, wherein the non-volatile oil is chosen from among isononyl isononanoate, dicaprylyl carbonate, diisostearyl malate, or one of the mixtures thereof.

28. The matte ipstick composition according to claim 20, wherein the oil is present at a content of between 40% and 80% by weight, preferably between 45% and 70% by weight, more preferentially between 50% and 60% by weight, with respect to the weight of the composition.

29. The matte lipstick composition according to claim 20, wherein the structuring agent is a wax chosen from among beeswax, synthetic wax, polyethylene wax or the mixture thereof.

30. The matte lipstick composition according to claim 29, wherein the wax is present in the cosmetic composition according to the invention at a content of between 10% and 30%, preferably between 15% and 25%, the percentages being percentages by weight with respect to the total weight of the composition.

31. The matte lipstick composition according to claim 20, wherein the composition further comprises an additional filler chosen from among starch, a clay of the mixture thereof.

32. The matte lipstick composition according to claim 20, wherein the colouring agent is chosen from among water-soluble or liposoluble colorants, pigments, nacres, lacquers or one of the mixtures thereof.

33. The matte lipstick composition according to claim 20, wherein the total content of colouring agent in the composition is between 5% and 8% by weight with respect to the total weight of the composition.

34. The matte lipstick composition according to claim 20, wherein the lipstick composition according to the invention spread on a contrast chart with a white background over a thickness of 500 microns thanks to an automatic spreader has a gloss value measured at 85° using a glossmeter named micro-TRI-gloss or PICOGLOSS 503 sold by the company BYK of less than 30, preferably less than 20, more preferably less than 10.

35. The matte lipstick composition according to claim 20, wherein the matte lipstick composition has a hardness measured at 20° C. using a texture analyser sold under the trade name “TA-XT Plus Microstable System” by the company Swantech of less than 300 g (grams), 1 day after manufacture and after storage of 1 day, 15 days, 1 month and 2 months in an oven at 45° C.

36. A matte lipstick composition comprising at least one structuring agent, at least one oil, at least one colouring agent, and at least one particular filler as a mattifying agent, wherein the particular filler serving as a mattifying agent is selected from among magnesium carbonate powders, said lipstick composition according to the invention spread on a contrast chart having a gloss value measured at 85° using a glossmeter known as micro-TRI-gloss or PICOGLOSS 503 sold by the company BYK of less than 10, and said composition having a hardness measured at 20° C. using a texture analyser sold under the trade name “TA-XT Plus Microstable System” by the company Swantech of less than 300 g (grams), 1 day after manufacture and after storage of 1 day, 15 days, 1 month and 2 months in an oven at 45° C.

37. A lipstick comprising the matte lipstick composition according to claim 20, wherein the lipstick comprises:
from 8% to 20% by weight of mattifying filler with respect to the total weight of the composition;
from 5% to 8% by weight of colouring agent with respect to the total weight of the composition;
from 10% to 30% and preferably from 15% to 25% by weight of structuring agent with respect to the total weight of the composition; and
from 40% to 80%, and preferably from 45% to 70% and more preferably from 50% to 60% of oil with respect to the total weight of the composition.

38. A method for preparing the matte lipstick composition according to claim 20 or a lipstick composition comprising the following steps:
grinding the pigments, if present, beforehand in a portion of the oil;
melting the structuring agent and the rest of the oil;
adding to the molten mixture obtained in (2), the particular filler as a mattifying agent, any colouring agents other than the pigments and any additional fillers as well as any pigments from step (1) under continuous stirring;
optionally adding the active substances or fragrances; and
pouring the hot composition obtained in step (4) into moulds, then allowing to cool until solidification.

39. A method of mattifying without hardening the matte lipstick composition according to claim 20 or a lipstick comprising said matte lipstick composition, said method comprising:
adding a particular filler as a mattifying agent selected from particles having a specific surface area of less than 30 m²/g.
